(19) United States
(12) Patent Application Publication
(10) Pub. No.: US 2010/0210646 A1
(43) Pub. Date: Aug. 19, 2010

(54) 2-MORPHOLIN-4-YL-PYRIMIDINES AS PI3K INHIBITORS

(75) Inventors: Paul John Goldsmith, Berkshire (GB); Timothy Colin Hancox, Berkshire (GB); Neil Anthony Pegg, Berkshire (GB); Stephen Joseph Shuttleworth, Berkshire (GB); Jonathan Martin Large, Surrey (GB); Edward McDonald, Surrey (GB)

Correspondence Address: VIKSNINS HARRIS & PADYS PLLP, P.O. BOX 111098, ST. PAUL, MN, 55111-1098, US

(73) Assignees: F. Hoffmann-La Roche AG, Basel (CH); The Institute of Cancer Research: Royal Cancer Hos pital, London (GB)

(21) Appl. No.: 12/594,552

(22) PCT Filed: Apr. 14, 2008
(86) PCT No.: PCT/GB08/01294

§ 371 Date: Apr. 21, 2010

(30) Foreign Application Priority Data
Apr. 12, 2007 (GB) 0707087.3

(51) Int. Cl. A61K 31/5377; C07D 413/14; A61P 35/00; A61P 37/00; A61P 31/12; A61P 29/00; A61P 25/00; A61P 9/00; A61P 5/00; A61P 3/00

(52) U.S. Cl. 514/235.2; 544/122

(57) ABSTRACT

The invention provides a pyrimidine compound of formula (I):

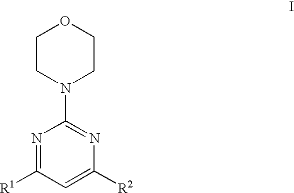

wherein R¹ and R² have any of the values defined herein; or a pharmaceutically acceptable salt thereof. These compounds are inhibitors of PI3K and may thus be used to treat diseases and disorders arising from abnormal cell growth, function or behaviour associated with PI3 kinase such as cancer, immune disorders, cardiovascular disease, viral infection, inflammation, metabolism/endocrine function disorders and neurological disorders.

### FIELD OF THE INVENTION

The present invention relates to pyrimidine compounds and to their use as inhibitors of phosphatidylinositol 3-kinase (PI3K).

### BACKGROUND TO THE INVENTION

Phosphatidylinositol (hereinafter abbreviated as “PI”) is one of a number of phospholipids found in cell membranes. In recent years it has become clear that PI plays an important role in intracellular signal transduction. In the late 1980s, a PI3 kinase (PI3K) was found to be an enzyme which phosphorylates the 3-position of the inositol ring of phosphatidylinositol (D. Whitman et al, 1988, Nature, 332, 664).

PI3K was originally considered to be a single enzyme, but it has now been clarified that a plurality of subtypes are present in PI3K. Each subtype has its own mechanism for regulating activity. Three major classes of PI3Ks have been identified on the basis of their in vitro substrate specificity (B. Vanhaesebroeck, 1997, Trend in Biol. Sci, 22, 267). Substrates for class I PI3Ks are PI, PI 4-phosphate (PI4P) and PI 4,5-biphosphate (PI (4,5)P2). Class I PI3Ks are further divided into two groups, class Ia and class Ib, in terms of their activation mechanism. Class Ia PI3Ks include PI3K p110α, p110β and p110δ subtypes, which transmit signals from tyrosine kinase-coupled receptors. Class Ib PI3K includes a p110γ subtype activated by a G protein-coupled receptor. PI and PI(4)P are known as substrates for class II PI3Ks. Class II PI3Ks include PI3K C2α, C2β and C2γ subtypes, which are characterized by containing C2 domains at the C terminus. The substrate for class III PI3Ks is PI only.

In the PI3K subtypes, the class Ia subtype has been most extensively investigated to date. The three subtypes of class Ia are heterodimers of a catalytic 110 kDa subunit and regulatory subunits of 85 kDa or 55 kDa. The regulatory subunits contain SH2 domains and bind to tyrosine residues phosphorylated by growth factor receptors with a tyrosine kinase activity or oncogene products, thereby inducing the PI3K activity of the p110 catalytic subunit which phosphorylates its lipid substrate. Thus, the class Ia subtypes are considered to be associated with cell proliferation and carcinogenesis, immune disorders and conditions involving inflammation.

WO 01/083456 describes a series of condensed heteroaryl derivatives which have activity as inhibitors of PI3 K and which suppress cancer cell growth.

### SUMMARY OF THE INVENTION

It has now been found that a series of novel pyrimidine compounds have activity as inhibitors of PI3K. The compounds exhibit selectivity for class Ia PI3Ks over class Ib, in particular for the p110δ subtype. Accordingly, the present invention provides a compound which is a pyrimidine of formula (I):

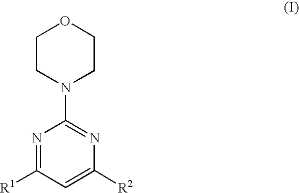

wherein

R¹ is a group —NR—(CHR)ₘ—X;

R² is a substituted indolyl group;

R is H or C₁-C₆ alkyl;

m is 1, 2, 3 or 4; and

X is a pyridyl ring;

or a pharmaceutically acceptable salt thereof.

### DETAILED DESCRIPTION OF THE INVENTION

A C₁-C₆ alkyl group is linear or branched. A C₁-C₆ alkyl group is typically a C₁-C₄ alkyl group, for example a methyl, ethyl, propyl, n-butyl, sec-butyl or tert-butyl group. A C₁-C₆ alkyl group is unsubstituted or substituted, typically by one or more groups Z or R⁷ as defined below. Typically it is C₁-C₄ alkyl, for example methyl, ethyl, i-propyl, n-propyl, t-butyl, s-butyl or n-butyl.

Z is selected from H, halo, —OR, —SR, CH₂OR, —CF₃, -(halo)-C₁-C₆ alkyl, —(C(R⁸)₂)_{q}O-(halo)-C₁-C₆ alkyl, —CO₂R, —(C(R⁸)₂)_{q}CO₂R, —(C(R⁸)₂)_{q}COR, CF₂OH, CH(CF₃)OH, C(CF₃)₂OH, —(CH₂)_{q}OR, —(C(R⁸)₂)_{q}OR, —(CH₂)_{q}NR₂, —(C(R⁸)₂)_{q}NR₂, —C(O)N(R)₂, —(C(R⁸)₂)_{q}CONR₂, —NR₂, —(C(R⁸)₂)_{q}NR₂, —NRC(O)R, —(C(R⁸)₂)_{q}NRC(O)OR, —S(O)ₘR, —S(O)ₘN(R)₂, —(C(R⁸)₂)_{q}S(O)ₘN(R)₂, —OC(O)R, —(C(R⁸)₂)_{q}OC(O)R, —OC(O)N(R)₂, —(C(R⁸)₂)_{q}OC(O)N(R)₂, —(C(R⁸)₂)_{q}OC(O)NR₂, —NRS(O)ₘR, —(C(R⁸)₂)_{q}NRS(O)ₘR, —NRC(O)N(R)₂, —(C(R⁸)₂)_{q}NRC(O)N(R)₂, CN, halogen, —NO₂ and a 5- to 12-membered aryl or heteroaryl group, which group is unsubstituted or substituted, wherein each R is independently selected from H, C₁-C₆ alkyl, C₃-C₁₀ cycloalkyl and a 5- to 12-membered aryl or heteroaryl group, the group being unsubstituted or substituted, m is 1 or 2 and q is 0, 1 or 2.

R⁷ is selected from C₁-C₆ alkoxy, OR⁸, SR⁸, S(O)ₘR⁸, nitro, CN, halogen, —C(O)R⁸, —CO₂R⁸, —C(O)N(R⁸)₂ and —N(R⁸)₂.

R⁸, each of which is the same or different when more than one is present in a given substituent, is selected from H, C₁-C₆ alkyl and C₃-C₁₀ cycloalkyl, and m is 1 or 2.

A halogen is F, Cl, Br or I. Preferably it is F, Cl or Br. A C₁-C₆ alkyl group substituted by halogen may be denoted by the term “halo-C₁-C₆ alkyl”, which means an alkyl group in which one or more hydrogens is replaced by halo. A halo-C₁-C₆ alkyl group preferably contains one, two or three halo groups. A preferred example of such a group is trifluoromethyl.

A pyridyl group is, for instance a pyrid-2-yl, pyrid-3-yl or pyrid-4-yl group.

R² is an indolyl group which is substituted. The indolyl group may be linked to the pyrimidine core via any available ring position. It may, for instance, be an indol-4-yl, indol-5-yl, indol-6-yl or indol-7-yl group.

The indolyl group may be substituted at one or more available ring positions. Typically it bears the substituent on the benzene moiety of the indole group. For instance, an indol-4-yl group is typically substituted at the 5-, 6- or 7-position, more typically at the 5- or 6-position. An indol-5-yl group is typically substituted at the 4-, 6- or 7-position, more typically at the 4- or 6-position. An indol-6-yl group is typically substituted at the 4-, 5- or 7-position, more typically at the 4- or 5-position. An indol-7-yl group is typically substituted at the 4-, 5- or 6-position, more typically at the 5- or 6-position.

Examples of suitable substituents for the indolyl group include CN, halo, —C(O)NR₂, perhalo(C₁-C₆)alkyl such as CF₃, —SO₂R, —SO₂NR₂, and a 5-membered heteroaryl group containing 1, 2, 3 or 4 heteroatoms selected from O, N and S, wherein R is H or C₁-C₆ alkyl. Typically the substituent is an electron-withdrawing group.

The 5-membered heteroaryl group may be, for example, furan, thiophene, pyrrole, imidazole, pyrazole, triazole, tetrazole, oxazole, isoxazole, oxadiazole, thiazole, isothiazole, or thiadiazole.

In one embodiment the substituted indolyl group is an indol-4-yl group substituted at the 5- or 6-position, in particular the 6-position, by CN, halo, —C(O)NH₂, —CF₃, —SO₂Me, —SO₂NMe₂ or a 5-membered heteroaryl group as defined above. Typically the indol-4-yl group is substituted at the 5- or 6-position by halo, in particular by F. More typically the indol-4-yl group is substituted at the 6-position by halo, in particular by F.

In one embodiment the pyrimidine is of formula (Ia):

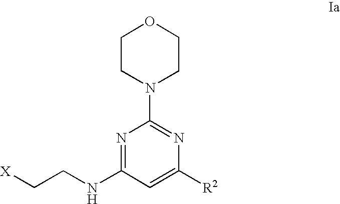

wherein X and R² are as defined above for formula (I).

In formula (I) or (Ia), X is typically a pyrid-3-yl or pyrid-4-yl group, in particular a pyrid-3-yl group. R² is typically an indol-4-yl group substituted at the 5-position by halo or at the 6-position by halo, CN, —CONH₂, —SO₂NMe₂ or —SO₂Me.

Specific examples of compounds of the invention include those listed in the following Table 1:

TABLE 1

| Compound | | |
|---|---|---|
| No. | Structure | Name |
| 1 | 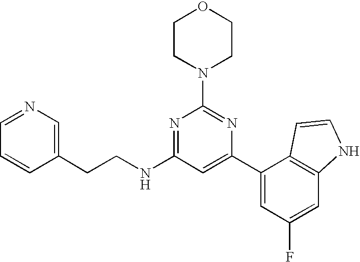 | [6-(6-Fluoro-1H-indol-4-yl)-2-morpholin-4-yl- pyrimidin-4-yl]-(2-pyridin-3-yl-ethyl)-amine |
| 2 | 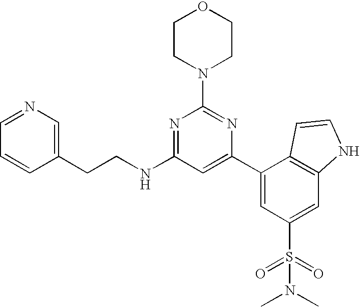 | 4-[2-morpholin-4-yl-6-(2-pyridin-3-yl- ethylamino)-pyrimidin-4-yl]-1H-indole-6- sulfonic acid dimethylamide. |
| 3 | 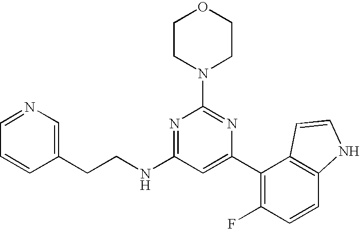 | [6-(5-Fluoro-1H-indol-4-yl)-2-morpholin-4-yl- pyrimidin-4-yl]-(2-pyridin-3-yl-ethyl)-amine |
| 4 | 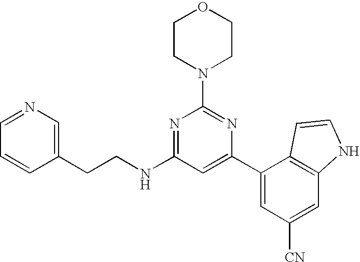 | 4-[2-Morpholin-4-yl-6-(2-pyridin-3-yl- ethylamino)-pyrimidin-4-yl]-1H-indole-6- carbonitrile |
| 5 | 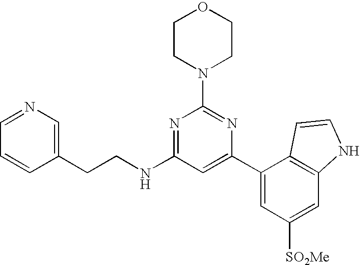 | [6-(6-Methanesulfonyl-1H-indol-4-yl)-2- morpholin-4-yl-pyrimidin-4-yl]-(2-pyridin-3-yl- ethyl)-amine |
| 6 | 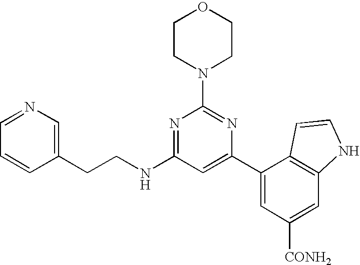 | 4-[2-Morpholin-4-yl-6-(2-pyridin-3-yl- ethylamino)-pyrimidin-4-yl]-1H-indole-6- carboxylic acid amide |
| 7 | 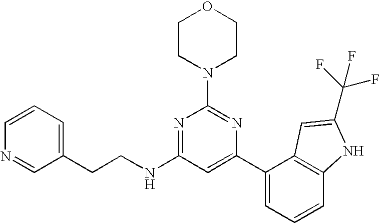 | [6-(2-Trifluoromethyl-1H-indol-4-yl)-2- morpholin-4-yl-pyrimidin-4-yl]-(2-pyridin-3-yl- ethyl)-amine |
| 8 | 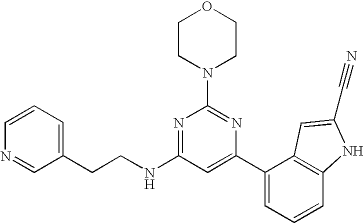 | [6-(2-Cyano-1H-indol-4-yl)-2-morpholin-4-yl- pyrimidin-4-yl]-(2-pyridin-3-yl-ethyl)-amine |

and the pharmaceutically acceptable salts thereof.

Pyrimidines of the invention may be produced by a process which comprises a palladium-mediated (Suzuki-type) cross-coupling reaction. Thus a pyrimidine of formula (I) may be produced by a process which comprises treating a compound of formula (II):

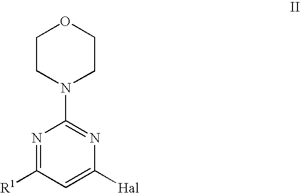

wherein R¹ is defined above and Hal is a halogen, with a boronic acid or ester thereof of formula R²B(OR¹⁵)₂, in which R² is as defined above and each R¹⁵ is H or C₁-C₆ alkyl or the two groups OR¹⁵ form, together with the boron atom to which they are attached, a pinacolato boronate ester group, in the presence of a Pd catalyst.

The intermediate compounds of formula (II) are known compounds which can be obtained commercially or made by routine synthetic chemical techniques. For example, a compound of formula (II) may be produced by a process which comprises treating a compound of formula (III):

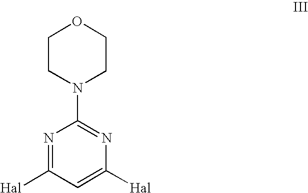

wherein each Hal is halogen, with an amine of formula HNR—(CHR)ₘ—X in a solvent in the presence of a base.

Pyrimidines of formula (I) may be converted into pharmaceutically acceptable salts, and salts may be converted into the free compound, by conventional methods. Pharmaceutically acceptable salts include salts of inorganic acids such as hydrochloric acid, hydrobromic acid and sulfuric acid, and salts of organic acids such as acetic acid, oxalic acid, malic acid, methanesulfonic acid, trifluoroacetic acid, benzoic acid, citric acid and tartaric acid. In the case of compounds of the invention bearing a free carboxy substituent, the salts include both the above-mentioned acid addition salts and the salts of sodium, potassium, calcium and ammonium. The latter are prepared by treating the free pyrimidine of formula (I), or the acid addition salt thereof, with the corresponding metal base or ammonia.

Compounds of the present invention have been found in biological tests to be inhibitors of PI3 kinase. The compounds are selective for class Ia PI3 kinases over class Ib. In general the compounds are selective for the p110δ isoform, for instance p110δ over p110γ.

A compound of the present invention may thus be used as an inhibitor of PI3 kinase, in particular of a class Ia PI3 kinase. Accordingly, a compound of the present invention can be used to treat a disease or disorder arising from abnormal cell growth, function or behaviour associated with PI3 kinase. Examples of such diseases and disorders are discussed by Drees et al in Expert Opin. Ther. Patents (2004) 14(5):703-732. These include proliferative disorders such as cancer, immune disorders, cardiovascular disease, viral infection, inflammation, metabolism/endocrine disorders and neurological disorders. Examples of metabolism/endocrine disorders include diabetes and obesity. Examples of cancers which the present compounds can be used to treat include leukaemia, brain tumours, renal cancer, gastric cancer and cancer of the skin, bladder, breast, uterus, lung, colon, prostate, ovary and pancreas.

A compound of the present invention may be used as an inhibitor of PI3 kinase. A human or animal patient suffering from a disease or disorder arising from abnormal cell growth, function or behaviour associated with PI3 kinase, such as an immune disorder, cancer, cardiovascular disease, viral infection, inflammation, a metabolism/endocrine disorder or a neurological disorder, may thus be treated by a method comprising the administration thereto of a compound of the present invention as defined above. The condition of the patient may thereby be improved or ameliorated.

A compound of the present invention can be administered in a variety of dosage forms, for example orally such as in the form of tablets, capsules, sugar- or film-coated tablets, liquid solutions or suspensions or parenterally, for example intramuscularly, intravenously or subcutaneously. The compound may therefore be given by injection or infusion.

The dosage depends on a variety of factors including the age, weight and condition of the patient and the route of administration. Daily dosages can vary within wide limits and will be adjusted to the individual requirements in each particular case. Typically, however, the dosage adopted for each route of administration when a compound is administered alone to adult humans is 0.0001 to 50 mg/kg, most commonly in the range of 0.001 to 10 mg/kg, body weight, for instance 0.01 to 1 mg/kg. Such a dosage may be given, for example, from 1 to 5 times daily. For intravenous injection a suitable daily dose is from 0.0001 to 1 mg/kg body weight, preferably from 0.0001 to 0.1 mg/kg body weight. A daily dosage can be administered as a single dosage or according to a divided dose schedule.

A compound of the invention is formulated for use as a pharmaceutical or veterinary composition also comprising a pharmaceutically or veterinarily acceptable carrier or diluent. The compositions are typically prepared following conventional methods and are administered in a pharmaceutically or veterinarily suitable form. The compound may be administered in any conventional form, for instance as follows:

A) Orally, for example, as tablets, coated tablets, dragees, troches, lozenges, aqueous or oily suspensions, liquid solutions, dispersible powders or granules, emulsions, hard or soft capsules, or syrups or elixirs. Compositions intended for oral use may be prepared according to any method known in the art for the manufacture of pharmaceutical compositions and such compositions may contain one or more agents selected from the group consisting of sweetening agents, flavouring agents, colouring agents and preserving agents in order to provide pharmaceutically elegant and palatable preparations.

Tablets contain the active ingredient in admixture with non-toxic pharmaceutically acceptable excipients which are suitable for the manufacture of tablets. These excipients may be for example, inert diluents, such as calcium carbonate, sodium carbonate, lactose, dextrose, saccharose, cellulose, corn starch, potato starch, calcium phosphate or sodium phosphate; granulating and disintegrating agents, for example, maize starch, alginic acid, alginates or sodium starch glycolate; binding agents, for example starch, gelatin or acacia; lubricating agents, for example silica, magnesium or calcium stearate, stearic acid or talc; effervescing mixtures; dyestuffs, sweeteners, wetting agents such as lecithin, polysorbates or lauryl sulphate. The tablets may be uncoated or they may be coated by known techniques to delay disintegration and adsorption in the gastrointestinal tract and thereby provide a sustained action over a longer period. For example, a time delay material such as glyceryl monostearate or glyceryl distearate may be employed. Such preparations may be manufactured in a known manner, for example by means of mixing, granulating, tableting, sugar coating or film coating processes.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is present as such, or mixed with water or an oil medium, for example, peanut oil, liquid paraffin, or olive oil.

Aqueous suspensions contain the active materials in admixture with excipients suitable for the manufacture of aqueous suspensions. Such excipients are suspending agents, for example, sodium carboxymethylcellulose, methylcellulose, hydroxypropylmethyl-cellulose, sodium alginate, polyvinylpyrrolidone gum tragacanth and gum acacia; dispersing or wetting agents may be naturally-occurring phosphatides, for example lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides for example polyoxyethylene sorbitan monooleate.

The said aqueous suspensions may also contain one or more preservatives, for example, ethyl or n-propyl p-hydroxybenzoate, one or more colouring agents, such as sucrose or saccharin.

Oily suspension may be formulated by suspending the active ingredient in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol.

Sweetening agents, such as those set forth above, and flavouring agents may be added to provide a palatable oral preparation. These compositions may be preserved by this addition of an antioxidant such as ascorbic acid. Dispersible powders and granules suitable for preparation of an aqueous suspension by the addition of water provide the active ingredient in admixture with a dispersing or wetting agent, a suspending agent and one or more preservatives. Suitable dispersing or wetting agents and suspending agents are exemplified by those already mentioned above. Additional excipients, for example sweetening, flavouring and colouring agents, may also be present.

The pharmaceutical compositions of the invention may also be in the form of oil-in-water emulsions. The oily phase may be a vegetable oil, for example olive oil or arachis oils, or a mineral oil, for example liquid paraffin or mixtures of these. Suitable emulsifying agents may be naturally-occurring gums, for example gum acacia or gum tragacanth, naturally occurring phosphatides, for example soy bean lecithin, and esters or partial esters derived from fatty acids an hexitol anhydrides, for example sorbitan mono-oleate, and condensation products of the said partial esters with ethylene oxide, for example polyoxyethylene sorbitan monooleate. The emulsion may also contain sweetening and flavouring agents. Syrups and elixirs may be formulated with sweetening agents, for example glycerol, sorbitol or sucrose. In particular a syrup for diabetic patients can contain as carriers only products, for example sorbitol, which do not metabolise to glucose or which only metabolise a very small amount to glucose.

Such formulations may also contain a demulcent, a preservative and flavouring and coloring agents.

B) Parenterally, either subcutaneously, or intravenously, or intramuscularly, or intrasternally, or by infusion techniques, in the form of sterile injectable aqueous or oleaginous suspensions. This suspension may be formulated according to the known art using those suitable dispersing of wetting agents and suspending agents which have been mentioned above. The sterile injectable preparation may also be a sterile injectable solution or suspension in a non-toxic paternally-acceptable diluent or solvent, for example as a solution in 1,3-butane diol.

Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition fatty acids such as oleic acid find use in the preparation of injectables.

C) By inhalation, in the form of aerosols or solutions for nebulizers.

D) Rectally, in the form of suppositories prepared by mixing the drug with a suitable non-irritating excipient which is solid at ordinary temperature but liquid at the rectal temperature and will therefore melt in the rectum to release the drug. Such materials are cocoa butter and poly-ethylene glycols.

E) Topically, in the form of creams, ointments, jellies, collyriums, solutions or suspensions.

The invention will be further described in the Examples which follow:

### EXAMPLES

### General Synthetic Procedures

The following general schemes 1 to 3 are referred to in the Reference Examples as Examples which follow:

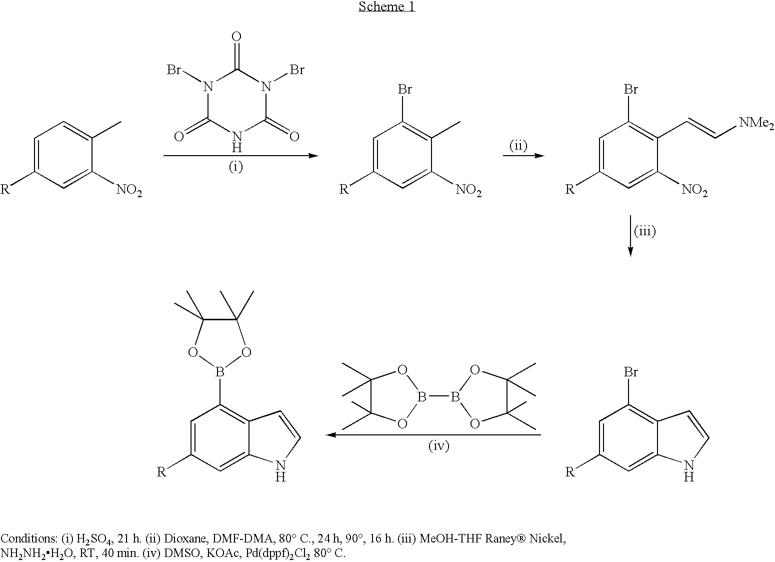

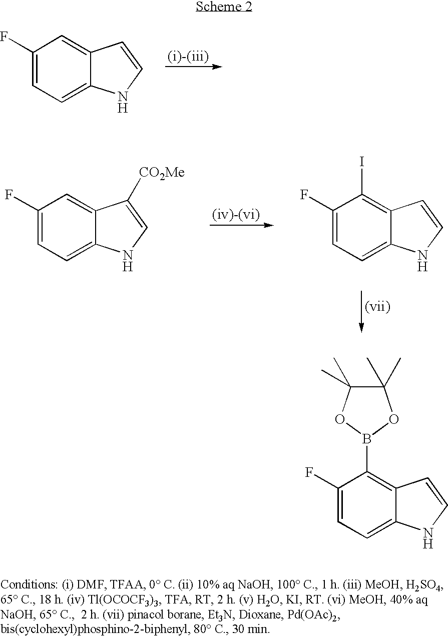

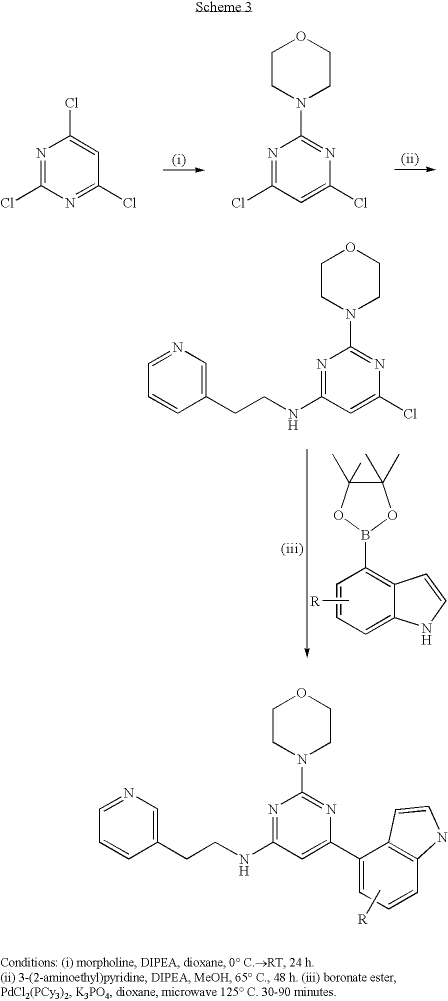

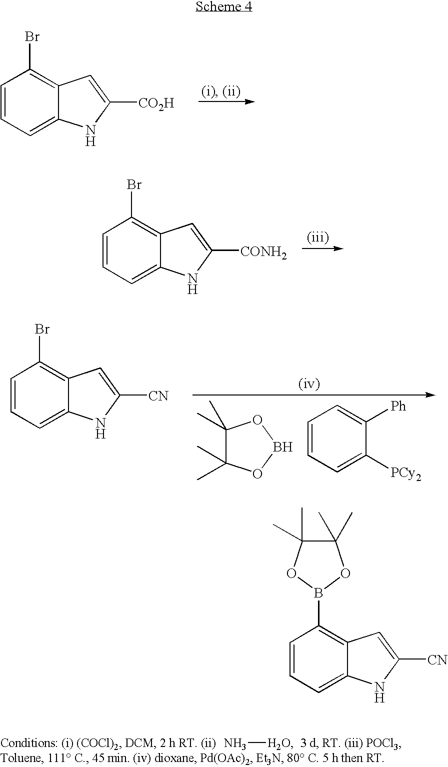

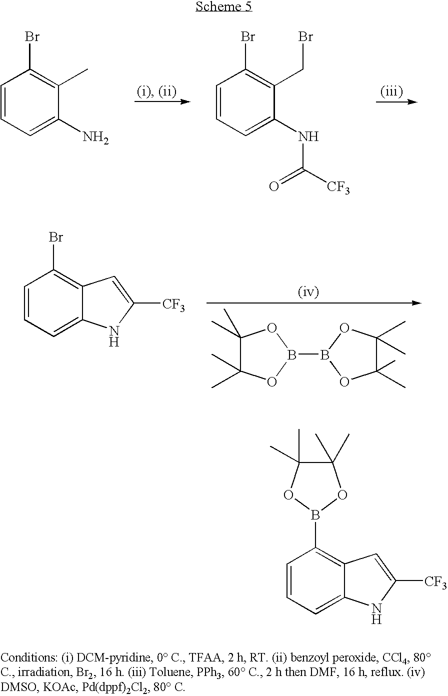

### General Experimental Details:

### NMR Spectrometry

NMR spectra were obtained on a Varian Unity Inova 400 spectrometer with a 5 mm inverse detection triple resonance probe operating at 400 MHz or on a Bruker Avance DRX 400 spectrometer with a 5 mm inverse detection triple resonance TXI probe operating at 400 MHz or on a Bruker Avance DPX 400 spectrometer with a 5 mm ¹H/¹³C Dual autotune probe operating at 400 MHz for ¹H or on a Bruker Avance DPX 300 spectrometer with a standard 5 mm dual frequency probe operating at 300 MHz. Shifts are given in ppm relative to tetramethylsilane @ 303K.

### Purification by Column Chromatography:

Compounds purified by column chromatography were purified using silica gel or Isolute® cartridge or Redisep® cartridge, eluting with gradients from 100-0 to 0-100% of cyclohexane/EtOAc, or from 100-0 to 0-100% pentane/EtOAc or from 100-0 to 70-30% DCM/MeOH (with or without the addition of NH₃ 0.1%). ‘Silica gel’ refers to silica gel for chromatography, 0.035 to 0.070 mm (220 to 440 mesh) (e.g. Fluka silica gel 60), and an applied pressure of nitrogen up to 10 p.s.i accelerated column elution. Where thin layer chromatography (TLC) has been used, it refers to silica gel TLC using plates, typically 3×6 cm silica gel on aluminium foil plates with a fluorescent indicator (254 nm), (e.g. Fluka 60778).

### Purification by Preparative HPLC:

Compounds purified by preparative HPLC were purified using either conditions A: Waters XBridge Prep Phenyl column (150×19 mm i.d. column with 5 μm particle size, PDA/MS detection, flow 21.25 ml/min), eluting with gradients from 95-5% to 5-95% water/acetonitrile containing 0.1% dimethylethylamine; or conditions B: C18-reverse-phase column (100×22.5 mm i.d. Genesis column with 7 μm particle size, UV detection at 230 or 254 nm, flow 5-15 mL/min), eluting with gradients from 100-0% to 0-100% water/acetonitrile or water/MeOH containing 0.1% TFA. When using conditions B the free base was liberated by partitioning between EtOAc and a sat. solution of sodium bicarbonate. The organic layer was dried (MgSO₄) and concentrated in vacuo. Alternatively, the free base was liberated by passing through an Isolute® SCX-2 cartridge, eluting with NH₃ in methanol.

### Abbreviations Used in the Experimental Section:

aq.=aqueous
BOC=t-Butoxycarbonyl
bs=broad singlet (NMR)
Cs₂CO₃=cesium carbonate
d=doublet (NMR)
DCM=dichloromethane
DIPEA=diisopropylethylamine
DMA=dimethylacetamide
DMAP=dimethylaminopyridine
DME=dimethoxyethane
DMF=dimethylformamide

### DMP=

DMSO=dimethylsulfoxide
eq.=equivalents
EtOAc=ethyl acetate
EtOH=ethanol
h=hour(s)
HATU=O-(7-Azabenzotriazol-1-yl)-N,N,N′,N′-tetramethyluronium hexafluorophosphate
HCl=hydrochloric acid
H₂O=water
HPLC=high pressure liquid chromatography
IMS=industrial methylated spirit
iPrOH=isopropanol
LCMS=liquid chromatography mass spectrometry
M=molar
m=multiplet (NMR)
MeOH=methanol
mg=milligram
MgSO₄=magnesium sulphate
min=minute(s)
mL=millilitre
Na₂CO₃=sodium carbonate
NaHCO₃=sodium hydrogen carbonate
NaOH=sodium hydroxide
Na₂SO₄=sodium sulfate
NMR=nuclear magnetic resonance
q=quartet (NMR)
Rt=retention time
RT=room temperature
sat=saturated
t=triplet (NMR)
TFA=trifluoroacetic acid
THF=tetrahydrofuran
TLC=thin layer chromatography

### Reference Example 1

### Formation of Boronate Ester

The boronate ester product of the final step of scheme 1 above was prepared as follows. To a solution of halide (1 eq.) and bis(pinacolato)diboron (1.3 eq.) in DMSO were added KOAc (3 eq.) and [1,1′-bis(diphenylphosphine)ferrocene]-dichloropalladium (0.05 eq.). The mixture was heated at 90° C. until completion of the reaction. The reaction mixture was partioned between EtOAc and H₂O. The organic layer was washed successively with H₂O and brine, dried over Na₂SO₄ and evaporated to dryness. The resultant residue was then purified by column chromatography.

### Reference Example 2

### 4-N,N-Trimethyl-3-nitro-benzenesulfonamide

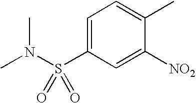

To a solution of dimethylamine in H₂O (40% w/w, 15.0 mL, 120 mmol) at 0° C. was added a solution of 4-methyl-3-nitro-benzenesulfonyl chloride (9.42 g, 40 mmol) in DCM (60 mL) over 30 min. The resulting mixture was stirred at 0° C. for 30 min before being allowed to warm to RT and stirred overnight. The reaction mixture was diluted with H₂O (100 mL) and DCM (40 mL), and the layers were separated. The organic layer was washed in succession with water, HCl (aq., 0.1 M) and brine before being dried over Na₂SO₄ and evaporated to dryness to give the title compound as a pale yellow solid (9.13 g, 94%).

[M+H]⁺ 244.9

### Reference Example 3

### 3-Bromo-4-N,N-trimethyl-5-nitro-benzenesulfonamide

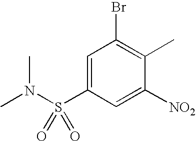

To a solution of 4-N,N-trimethyl-3-nitro-benzenesulfonamide (8.57 g, 34.7 mmol) in concentrated sulfuric acid (80 mL) was added 1,3-dibromo-[1,3,5]triazinane-2,4,6-trione (5.97 g, 20.8 mmol) and the orange reaction mixture was stirred at RT for 16 h. A further 2 g of 1,3-dibromo-[1,3,5]triazinane-2,4,6-trione was added and stirring continued for 5 h. The reaction mixture was then poured onto ice and water and stirred for 15 min. The resulting milky/white solid was filtered and washed with H₂O, before being dissolved in EtOAc. The organic layer was dried over Na₂SO₄ and evaporated to dryness to give the title compound as a white solid (10.41 g, 93%).

[M+H]⁺ 323.1 (⁷⁹Br) 325.0 (⁸¹Br)

### Reference Example 4

### 1-Bromo-5-methanesulfonyl-2-methyl-3-nitro-benzene

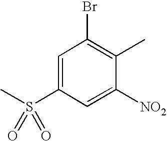

Prepared according to the method used in the preparation of 3-bromo-4-N,N-trimethyl-5-nitro-benzenesulfonamide using 4-methanesulfonyl-1-methyl-2-nitro-benzene in place of 4-N,N-trimethyl-3-nitro-benzenesulfonamide. The title compound was obtained as a white solid (17.0 g, 85%).

[M+H]⁺ 294.1 (⁷⁹Br) 296.0 (⁸¹Br)

### Reference Example 5

### 1-Bromo-5-fluoro-2-methyl-3-nitro-benzene

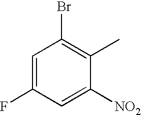

Prepared according to the method used in the preparation of 3-bromo-4-N,N-trimethyl-5-nitro-benzenesulfonamide using 4-fluoro-1-methyl-2-nitro-benzene in place of 4-N,N-trimethyl-3-nitro-benzenesulfonamide. The title compound was obtained as a yellow solid (68.0 g, 79%).

NMR δ_{H} (300 MHz, CDCl₃) 2.59 (s, 3H), 7.50 (dd, J=2.8, 7.6, 1H) and 7.58 (dd, J=2.9, 7.4, 1H).

### Reference Example 6

### 4-Bromo-1H-indole-6-sulfonic acid dimethylamide

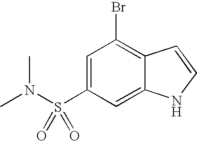

To a solution of 3-bromo-4-N,N-trimethyl-5-nitro-benzenesulfonamide (9.15 g, 28.3 mmol) in dioxane (60 mL) was added DMF-DMA (11.3 mL, 84.9 mmol). The deep red reaction mixture was heated at 80° C. for 24 h followed by heating at 90° C. for 16 h. The mixture was cooled to RT and concentrated to 50% of the volume, poured into H₂O and extracted into EtOAc. The organic layer was isolated and washed with H₂O, then brine, dried over Na₂SO₄, and evaporated to dryness to give 3-bromo-4-(2-dimethylamino-vinyl)-N,N-dimethyl-5-nitro-benzenesulfonamide as a red solid (10.4 g, 91%). To a suspension of the amide (10.4 g, 25.7 mmol) and Raney®-Nickel (suspension in H₂O, 20 mL) in MeOH:THF (1:1, 200 mL) was added hydrazine monohydrate (1.9 mL, 38.6 mmol) at 0° C. and the mixture stirred at RT for 40 min. The reaction mixture was then filtered through Celite and the filter cake washed with EtOAc and H₂O. The aqueous layer was isolated and then extracted with EtOAc. The combined organic layers were washed with H₂O, followed by brine, dried over Na₂SO₄ then evaporated to dryness. The resulting pink solid was purified by column chromatography, and subsequently recrystallised from iPrOH and EtOH to give the title compound as a white solid (3.5 g, 41%).

NMR δ_{H} (400 MHz, CDCl₃) 2.72 (s, 6H), 6.70 (m, 1H), 7.49 (apparent t, J=2.7, 1H), 7.68 (d, J=1.1, 1H), 7.94 (m, 1H) and 9.04 (bs, 1H).

### Reference Example 7

### 4-Bromo-6-methanesulfonyl-1H-indole

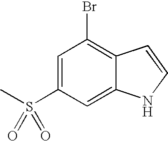

Prepared according to the method used in the preparation of 4-bromo-1H-indole-6-sulfonic acid dimethylamide using 1-bromo-5-methanesulfonyl-2-methyl-3-nitro-benzene in place of 3-bromo-4-N,N-trimethyl-5-nitro-benzenesulfonamide. The title compound was obtained as a white solid (1.8 g, 76%).

NMR δ_{H} (300 MHz, CDCl₃) 3.11 (s, 3H), 6.70 (m, 1H), 7.52 (dd, J=2.5, 3.0, 1H), 7.81 (d, J=1.5, 1H), 8.10 (dd, J=1.0, 1.5, 1H) and 9.34 (bs, 1H).

### Reference Example 8

### 4-Bromo-6-fluoro-1H-indole

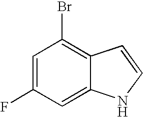

Prepared according to the method used in the preparation of 4-bromo-1H-indole-6-sulfonic acid dimethylamide using 1-bromo-5-fluoro-2-methyl-3-nitro-benzene in place of 3-bromo-4-N,N-trimethyl-5-nitro-benzenesulfonamide. The title compound was obtained as a white solid (6.06 g, 33%).

NMR δ_{H} (300 MHz, CDCl₃) 6.57 (apparent t, J=2.7, 1H), 7.04 (dd, J=2.1, 9.1, 1H), 7.12 (dd, J=2.1, 9.1, 1H), 7.20-7.25 (m, 1H) and 8.25 (s, 1H).

### Reference Example 9

### 4-(4,4,5,5-Tetramethyl-[1,3,2]dioxaborolan-2-yl)-1H-indole-6-carboxylic acid amide

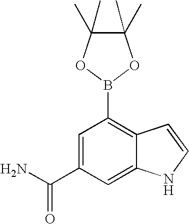

A solution of 4-bromo-1H-indole-6-carbonitrile (1 g, 4.50 mmol) in methanol (10 mL) was treated with 30% aqueous hydrogen peroxide (2.7 mL, 4.95 mmol) and a 1 M aqueous sodium hydroxide solution (5 mL) then heated at 40° C. for 1 h. The reaction mixture was cooled, treated with water and cooled in an ice-bath. The resulting precipitate was collected by filtration, washed with water and dried in vacuo to obtain 4-bromo-1H-indole-6-carboxylic acid amide (1.05 g, 97%), which was transformed into the title boronic ester by the general method (Scheme 1) (0.80 g, 67%).

NMR δ_{H} (300 MHz, DMSO-d₆) 1.35 (s, 12H), 6.78 (m, 1H), 7.10 (s, 1H), 7.51-7.54 (m, 1H), 7.94-7.97 (m, 2H), 8.06 (s, 1H) and 11.40 (bs, 1H).

### Reference Example 10

### 5-Fluoro-4-(4,4,5,5-tetramethyl-[1,3,2]dioxaborolan-2-yl)-1H-indole

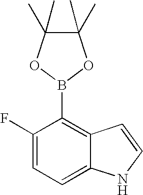

A solution of 5-fluoroindole (5 g, 37.0 mmol) in DMF (40 mL) was treated at 0° C. with trifluoroacetic anhydride (6.1 mL, 42.6 mmol). After 30 min, the reaction was poured into water and the resulting precipitate collected by filtration, washed with water, then dried in vacuo. The solid was then dissolved in 10% aqueous NaOH (200 mL) and heated at reflux for 1 h. The reaction mixture was then cooled, washed with dichloromethane and acidified with aqueous HCl. The resulting white precipitate was collected by filtration, washed with water, taken up in dichloromethane, washed with water, dried (MgSO₄) and evaporated in vacuo. The resulting material (5 g, 75%) was dissolved in methanol (80 mL) and treated with concentrated sulphuric acid (2 mL) then heated at reflux overnight. The reaction was cooled and the resulting precipitate collected, washed with water and evaporated in vacuo to give 5-fluoro-1H-indole-3-carboxylic acid methyl ester as a peach-coloured solid (4.5 g, 83%).

A solution of thallium tris(trifluoroacetate) (8.45 g, 15.6 mmol) in TFA (35 mL) was added to a solution of 5-fluoro-1H-indole-3-carboxylic acid methyl ester (2 g, 10.4 mmol) in TFA (10 mL) at room temperature and stirred for 2 h. The reaction mixture was evaporated in vacuo and the resulting residue suspended in water (25 mL) before being treated with a solution of potassium iodide (5.2 g, 31.3 mmol) in water (50 mL). The reaction mixture was treated with dichloromethane (100 mL) and methanol (5 mL) and the resulting precipitate removed by filtration through celite. The organic layer was separated, washed successively with sodium thiosulfate solution and brine, then dried (MgSO₄) and evaporated in vacuo. The resultant material was dissolved in methanol (60 mL) and treated with 40% aqueous NaOH solution (60 mL) then refluxed for 2 h. The reaction mixture was cooled and extracted with DCM/MeOH (ratio 95:5), dried (MgSO₄), filtered and evaporated in vacuo to give a crude solid. Purification by column chromatography gave 5-fluoro-4-iodo-1H-indole as a pale brown solid (1.05 g, 39%).

NMR δ_{H} (300 MHz, CDCl₃) 6.49-6.52 (m, 1H), 6.95 (apparent dt, J=0.4, 8.6, 1H), 7.26-7.33 (m, 2H) and 8.35 (s, 1H).

A solution of 5-fluoro-4-iodo-1H-indole (261 mg, 1.0 mmol) in dioxane (1 mL) was treated with triethylamine (0.2 mL, 1.4 mmol), palladium acetate (4.5 mg, 0.02 mmol) and bis(cyclohexyl)phosphino-2-biphenyl (28 mg, 0.08 mmol) then heated to 80° C. A solution of pinacolborane (1 M in THF, 2.66 mL, 2.66 mmol) was added via syringe. After 30 min, the reaction mixture was cooled, then diluted with water (10 mL) and DCM (10 mL). The resulting mixture was passed through a phase separation cartridge, and the dichloromethane layer was evaporated in vacuo to obtain the title compound which was used without further purification.

### Reference Example 11

### (6-Chloro-2-morpholin-4-yl-pyrimidin-4-yl)-(2-pyridin-3-yl-ethyl)-amine

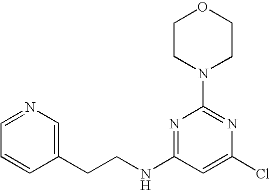

To a stirred solution of 2,4,6-trichloropyrimidine (10 ml; 87 mmol), and DIPEA (16 mL; 92 mmol) in dioxane (60 mL) at 5° C. was added morpholine (8 ml; 91 mmol) over 5 minutes (a white solid separates during addition). The reaction mixture was stirred whilst allowing to warm to r.t. overnight (16 h). Volatiles were removed in vacuo, the resulting residue was redissolved (CH₂Cl₂) and evaporated onto silica and purified by flash chromatography (90:10 to 50:50 petrol/EtOAc as eluent) to afford the regioisomeric products: 4-(4,6-dichloro-pyrimidin-2-yl)-morpholine (2.46 g; 12%); and 4-(2,6-dichloro-pyrimidin-4-yl)-morpholine (9.72 g; 48%).

A stirred solution of 4-(4,6-dichloro-pyrimidin-2-yl)-morpholine (0.50 g; 2.13 mmol), DIPEA (408 μL; 2.34 mmol) and 3-(2-aminoethyl)pyridine (290 mg; 2.37 mmol) in anhydrous methanol (10 mL) was heated at 65-70° C. for 48 h. The reaction mixture was partitioned between water/CH₂Cl₂, the organic layer was dried, concentrated and purified by flash chromatography (95:5 to 85:15 CH₂Cl₂/MeOH as eluent) to afford the title compound as a white solid (0.51 g; 75%).

δ_{H} (400 MHz, CDCl₃) 2.94 (t, J=6.8, 2H), 3.58-3.62 (m, 2H), 3.74-3.78 (m, 8H), 4.69 (br s, 1H), 5.71 (s, 1H), 7.26-7.28 (m, 1H), 7.53 (d, J=8.0, 1H), 8.50 (s, 1H), 8.52-8.53 (m, 1H).

### Reference Example 12

### 4-(4,4,5,5-Tetramethyl-[1,3,2]dioxaborolan-2-yl)-1H-indole-6-carbonitrile

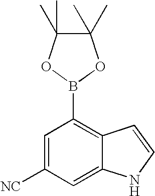

Prepared using the general method of Scheme 1. The title compound was obtained as an off-white solid.

δH (400 MHz, CDCl₃) 1.40 (s, 12H), 7.12 (m, 1H), 7.46 (t, J=2.9, 1H), 7.8 (t, J=1.1, 1H), 7.87 (d, J=1.3, 1H), 8.42 (br s, 1H).

### Reference Example 13

### 4-(4,4,5,5-Tetramethyl-[1,3,2]dioxaborolan-2-yl)-1H-indole-6-sulfonic acid dimethylamide

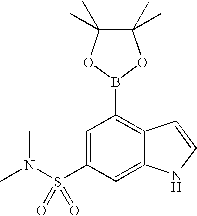

Prepared using the general method of Scheme 1. The title compound was obtained as a white solid (1.85 g, 46%).

[M+H]⁺ 350.2 (¹⁰B) 351.2 (¹¹B)

### Reference Example 14

### 4-(4,4,5,5-Tetramethyl-[1,3,2]dioxaborolan-2-yl)-6-trifluoromethyl-1H-indole

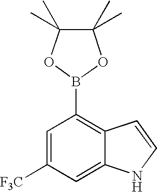

Prepared by using the general method of Scheme 1. The title compound was obtained as a pale yellow solid (1.37 g, 92%).

[M+H]⁺ 311.2 (¹⁰B) 312.2 (¹¹B)

### Reference Example 15

### 6-Methanesulfonyl-4-(4,4,5,5-tetramethyl-[1,3,2]dioxaborolan-2-yl)-1H-indole

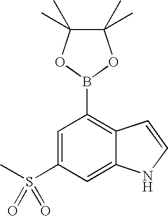

Prepared using the general method of Scheme 1. The title compound was obtained as a pale yellow solid (2.4 g, 51%).

NMR δ_{H} (300 MHz, DMSO-d₆) 1.36 (s, 12H), 3.18 (s, 3H), 6.87 (m, 1H), 7.73 (apparent t, J=2.5, 1H), 7.85 (d, J=1.5, 1H), 8.07 (dd, J=1.0, 1.5, 1H) and 11.73 (bs, 1H).

### Reference Example 16

### 6-Fluoro-4-(4,4,5,5-tetramethyl-[1,3,2]dioxaborolan-2-yl)-1H-indole

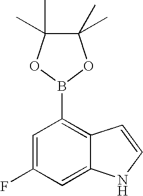

Prepared by using the general method of Scheme 1. The title compound was obtained as a white solid (4.6 g, 61%).

NMR δ_{H} (300 MHz, CDCl₃) 1.39 (s, 12H), 7.02 (m, 1H), 7.14-7.19 (m, 1H), 7.20-7.26 (m, 1H), 7.38 (dd, J=2.4, 9.9, 1H) and 8.16 (s, 1H).

### Reference Example 17

### 4-Bromo-1H-indole-2-carboxylic acid amide

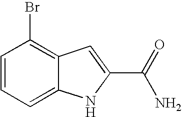

Oxalyl chloride (0.9 mL, 10 mmol) was added to a suspension of 4-bromo-1H-indole-2-carboxylic acid (2.1 g, 8.8 mmol) in DCM and the mixture was stirred for 2 h. The solution formed was added drop-wise to a stirring mixture of ammonia (37%, 50 mL) and ice (50 mL). The resulting mixture was allowed to stand for 3 days. The mixture was filtered and the filtrate extracted with EtOAc. The solid from the filtration was dissolved in EtOAc and the organic solutions were combined, dried (MgSO₄) and then evaporated to afford the title compound as a brown solid (2.1 g, 100%).

NMR δ_{H} (400 MHz, CD₃OD) 7.11 (dd, J=7.5, 8.3, 1H), 7.16 (d, J=0.9, 1H), 7.25 (dd, J=0.78, 7.54, 1H) and 7.43 (d, J=8.3, 1H).

### Reference Example 18

### 4-Bromo-1H-indole-2-carbonitrile

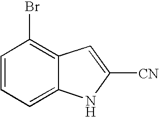

Phosphorous oxychloride (1.9 mL, 20 mmol) was added to a suspension of 4-bromo-1H-indole-2-carboxylic acid amide (1.32 g, 5.5 mmol.) in toluene (10 mL) and the mixture was stirred at reflux for 45 min. On cooling, the mixture was poured into an aqueous Na₂CO₃ solution (sat., 50 mL) and the mixture stirred until effervescence had subsided. The layers were separated, the aqueous phase extracted with EtOAc and the combined organic layers dried (MgSO₄) and evaporated to dryness. The crude material was purified by column chromatography to afford the title compound as a solid (1.00 g, 82%).

NMR δ_{H} (400 MHz, CDCl₃) 7.22-7.28 (m, 2H), 7.35-7.40 (m, 2H) and 8.79 (s, 1H).

### Reference Example 19

### 4-Bromo-2-trifluoromethyl-1H-indole

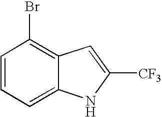

A solution of 2-methyl-3-bromo-aniline (6.05 g, 37 mmol) in pyridine (8 mL) and DCM (150 mL) was cooled to 0° C. and treated drop-wise with trifluoroacetic anhydride (11.5 mL, 81.4 mmol). The reaction mixture was stirred at RT for 2 h, then quenched with an aqueous solution of ammonium chloride. The organic layer was dried over MgSO₄, and evaporated to dryness to give N-(3-bromo-2-methyl-phenyl)-2,2,2-trifluoro-acetamide as an off-white solid, which was used without further purification (10 g).

NMR δ_{H} (400 MHz, CDCl₃) 2.38 (s, 3H), 7.14 (apparent t, J=8.0, 1H), 7.53 (d, J=8.0, 1H), 7.66 (d, J=8.0, 1H) and 7.75 (bs, 1H).

A solution of N-(3-bromo-2-methyl-phenyl)-2,2,2-trifluoro-acetamide (2.1 g, 7.4 mmol) and benzoyl peroxide (100 mg) in carbon tetrachloride (50 mL) was heated to reflux under irradiation (150 W tungsten lamp). A solution of bromine (0.55 mL, 10.4 mmol) in carbon tetrachloride (3 mL) was then added drop-wise to the refluxing solution, and heating was pursued for 16 h. The reaction mixture was left to cool to RT and diluted with DCM. The organic layer was washed with sodium thiosulfate, and evaporated to dryness to give N-(3-bromo-2-bromomethyl-phenyl)-2,2,2-trifluoro-acetamide as a brown residue which was used without further purification (2.9 g).

NMR δ_{H} (400 MHz, CDCl₃) 4.71 (s, 2H), 7.30 (apparent t, J=8.0, 1H), 7.55 (d, J=8.0, 1H), 7.82 (d, J=8.0, 1H) and 8.79 (bs, 1H).

A solution of N-(3-bromo-2-bromomethyl-phenyl)-2,2,2-trifluoro-acetamide (2.9 g) in toluene (40 mL) was treated with triphenylphosphine (2.3 g, 8.7 mmol). The solution was stirred at 60° C. for 2 h, then cooled to 0° C. The beige solid that precipitated was collected by filtration, washed with diethyl ether, then dissolved in DMF (60 mL), and heated to reflux under nitrogen for 16 h. The reaction mixture was evaporated to dryness, then partitioned between EtOAc and a sat. sodium carbonate solution. The organic layer was isolated, dried (MgSO₄), and purified by column chromatography to give the title compound as a yellow solid (1.55 g, 84%).

NMR δ_{H} (400 MHz, CDCl₃) 7.00 (s, 1H), 7.19 (apparent t, J=7.9, 1H), 7.36-7.41 (m, 2H) and 8.53 (bs, 1H).

### Reference Example 20

### 4-(4,4,5,5-Tetramethyl-[1,3,2]dioxaborolan-2-yl)-2-trifluoromethyl-1H-indole

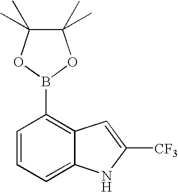

Prepared using the general method of Reference Example 1. The title compound was obtained as a white solid (1.5 g, 55%).

NMR δ_{H} (400 MHz, CDCl₃) 1.40 (s, 12H), 7.33 (dd, J=7.0, 8.3, 1H), 7.42 (s, 1H), 7.53 (d, J=8.3, 1H), 7.70 (d, J=7.0, 1H) and 8.37 (bs, 1H).

### Reference Example 21

### 4-(4,4,5,5-Tetramethyl-[1,3,2]dioxaborolan-2-yl)-1H-indole-2-carbonitrile

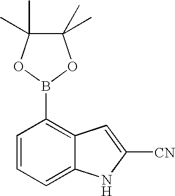

4,4,5,5-Tetramethyl-[1,3,2]dioxaborolane (2.1 mL, 14.5 mmol) was added drop-wise to a mixture of 4-bromo-1H-indole-2-carbonitrile (1.27 g, 5.8 mmol), palladium acetate (33 mg, 0.145 mmol), triethylamine (1.21 mL, 8.7 mmol) and 2-(dicyclohexylphosphino)biphenyl (203 mg, 0.58 mmol) in dioxane at 80° C. The reaction mixture was stirred at 80° C. for 5 h then allowed to stand at RT overnight. The reaction mixture was diluted with DCM and washed with water, then the organic layer was isolated, dried (MgSO₄) then concentrated in vacuo. The resultant crude material was purified by column chromatography to afford the title compound as a brown solid (1.02 g, 66%).

NMR δ_{H} (400 MHz, CDCl₃) 1.40 (s, 12H), 7.36-7.42 (m, 1H), 7.51 (apparent dt, J=1.0, 8.3, 1H), 7.67-7.74 (m, 2H) and 8.51 (s, 1H).

### Example 1

### [6-(6-Fluoro-1H-indol-4-yl)-2-morpholin-4-yl-pyrimidin-4-yl]-(2-pyridin-3-yl-ethyl)-amine

A mixture of (6-chloro-2-morpholin-4-yl-pyrimidin-4-yl)-(2-pyridin-3-yl-ethyl)-amine (88 mg, 0.28 mmol), 1 M aqueous Na₂CO₃ (0.82 mL, 3 eq.), indole boronate ester (129 mg, 1.8 eq.) and dichlorobis(triphenylphosphine) palladium (II) (10 mg, 0.05 eq.) in acetonitrile (3 mL) was heated for 50 minutes in a microwave reactor at 140° C. The mixture was partitioned between water and dichloromethane, the combined organic layers washed with brine, separated and dried (MgSO₄). The crude product was purified by column chromatography to give the desired compound as a white solid (20 mg).

δ_{H} (400 MHz, CDCl₃) 3.00 (t, J=6.8, 2H), 3.71 (q, J=6.8, 2H), 3.81-3.83 (m, 4H), 3.89-3.92 (m, 4H), 4.72 (br s, 1H), 6.21 (s, 1H), 7.04 (s, 1H), 7.15 (d, J=8.8, 1H), 7.38 (dd, J=10.6 and 2.2, 1H), 7.58 (d, J=8.0, 1H), 8.26 (br s, 1H), 8.53-8.55 (m, 2H).

[M+H]⁺ 419.2

### Example 2

### 4-[2-morpholin-4-yl-6-(2-pyridin-3-yl-ethylamino)-pyrimidin-4-yl]-1H-indole-6-sulfonic acid dimethylamide

Prepared using the method described for [6-(6-fluoro-1H-indol-4-yl)-2-morpholin-4-yl-pyrimidin-4-yl]-(2-pyridin-3-yl-ethyl)-amine to give the title compound as a white solid (0.024 g).

δ_{H} (400 MHz, CDCl₃) 2.74 (s, 6H), 3.00 (m, 2H), 3.74 (m, 2H), 3.82 (m, 2H), 3.90 (m, 4H), 4.77 (br s, 1H), 6.21 (s, 1H), 7.22 (m, 1H), 7.30 (m, 1H), 7.51 (m, 1H), 7.60 (m, 1H), 7.89 (m, 1H), 7.95 (s, 1H), 8.54 (m, 2H), 8.69 (br s, 1H).

[M+H]⁺ 508.2

### Example 3

### [6-(5-Fluoro-1H-indol-4-yl)-2-morpholin-4-yl-pyrimidin-4-yl]-(2-pyridin-3-yl-ethyl)-amine

Prepared using the method described for [6-(6-fluoro-1H-indol-4-yl)-2-morpholin-4-yl-pyrimidin-4-yl]-(2-pyridin-3-yl-ethyl)-amine to give a white solid (17 mg).

δ_{H} (400 MHz, CDCl₃) 2.89 (t, J=6.8, 2H), 3.59 (q, J=6.8, 2H), 3.70-3.73 (m, 4H), 3.77-3.79 (m, 4H), 4.61 (br s, 1H), 6.13 (d, J=2.8, 1H), 6.90-6.95 (m, 2H), 7.16-7.21 (m, 2H), 7.26 (dd, J=8.8 and 4.0, 1H), 7.48 (d, J=7.6, 1H), 8.12 (br s, 1H), 8.42-8.45 (m, 2H).

[M+H]⁺ 419.

### Example 4

### 4-[2-Morpholin-4-yl-6-(2-pyridin-3-yl-ethylamino)-pyrimidin-4-yl]-1H-indole-6-carbonitrile

Prepared using the method described for [6-(6-fluoro-1H-indol-4-yl)-2-morpholin-4-yl-pyrimidin-4-yl]-(2-pyridin-3-yl-ethyl)-amine to give an off-white solid (47 mg).

δ_{H} (400 MHz, CDCl₃) 3.00 (t, J=6.8, 2H), 3.73 (q, J=6.8, 2H), 3.81-3.83 (m, 4H), 3.89-3.91 (m, 4H), 4.77 (br s, 1H), 6.18 (s, 1H), 7.15 (s, 1H), 7.28-7.30 (m, 1H), 7.49-7.51 (m, 1H), 7.59 (d, J=7.6, 1H), 7.78 (s, 1H), 7.81 (s, 1H), 8.53-8.55 (m, 2H), 8.61 (br s, 1H).

[M+H]⁺ 426.

### Example 5

### [6-(6-Methanesulfonyl-1H-indol-4-yl)-2-morpholin-4-yl-pyrimidin-4-yl]-(2-pyridin-3-yl-ethyl)-amine

Prepared using the method described for [6-(6-fluoro-1H-indol-4-yl)-2-morpholin-4-yl-pyrimidin-4-yl]-(2-pyridin-3-yl-ethyl)-amine to give a white solid (17 mg).

δ_{H} (400 MHz, CDCl₃) 3.00 (t, J=6.8, 2H), 3.12 (s, 3H), 3.73 (q, J=6.8, 2H), 3.81-3.83 (m, 4H), 3.88-3.90 (m, 4H), 4.75 (br s, 1H), 6.22 (s, 1H), 7.02 (s, 1H), 7.26-7.28 (m, 1H), 7.54-7.55 (m, 1H), 7.59 (d, J=7.2, 1H), 8.05 (s, 1H), 8.11 (s, 1H), 8.53-8.55 (m, 2H), 8.65 (br s, 1H).

[M+H]⁺ 479.

### Example 6

### 4-[2-Morpholin-4-yl-6-(2-pyridin-3-yl-ethylamino)-pyrimidin-4-yl]-1H-indole-6-carboxylic acid amide

Prepared using the method described for [6-(6-fluoro-1H-indol-4-yl)-2-morpholin-4-yl-pyrimidin-4-yl]-(2-pyridin-3-yl-ethyl)-amine to give an off-white solid (14 mg).

δ_{H} (400 MHz, 95:5 CDCl₃/MeOD) 2.93 (t, J=6.8, 2H), 3.33 (s, 2H), 3.64 (t, J=6.8, 2H), 3.76-3.78 (m, 4H), 3.80-3.82 (m, 4H), 6.21 (s, 1H), 6.98 (s, 1H), 7.23-7.28 (m, 1H), 7.38-7.39 (m, 1H), 7.58 (d, J=7.6, 1H), 7.85 (s, 1H), 7.97 (s, 1H), 8.37-8.38 (m, 1H), 8.41 (s, 1H).

[M+H]⁺ 444.

### Example 7

### [6-(2-Trifluoromethyl-1H-indol-4-yl)-2-morpholin-4-yl-pyrimidin-4-yl]-(2-pyridin-3-yl-ethyl)-amine

Prepared using the method described for [6-(6-fluoro-1H-indol-4-yl)-2-morpholin-4-yl-pyrimidin-4-yl]-(2-pyridin-3-yl-ethyl)-amine to give a pale yellow solid (28 mg)

δ_{H} (400 MHz, CDCl₃) 2.99 (m, 3H); 3.50 (m, 2H); 3.82 (4H, m); 3.91 (4H, m); 4.73 (brs, 1H); 6.16 (s, 1H); 7.26 (m, 1H); 7.40 (m, 1H); 7.49 (m, 2H); 7.58 (m, 2H); 8.52-8.55 (m, 3H).

[M+H]⁺ 469.

### Example 8

### [6-(2-Cyano-1H-indol-4-yl)-2-morpholin-4-yl-pyrimidin-4-yl]-(2-pyridin-3-yl-ethyl)-amine

Prepared using the method described for [6-(6-fluoro-1H-indol-4-yl)-2-morpholin-4-yl-pyrimidin-4-yl]-(2-pyridin-3-yl-ethyl)-amine to give an off-white solid (24 mg).

δ_{H} (400 MHz, CDCl₃) 3.00 (m, 2H); 3.74 (m, 2H); 3.83 (m, 4H); 3.89 (m, 4H); 4.77 (brs, 1H); 6.15 (s, 1H); 7.31 (m, 1H); 7.46 (m, 2H); 7.59 (m, 2H); 8.55 (m, 2H); 9.02 (brs, 1H).

[M+H]⁺ 426

### Example 9

### Biological Testing

Compounds of the invention, prepared as described in the preceding Examples, were submitted to the following series of biological assays:

### (i) PI3K Biochemical Screening

Compound inhibition of PI3K was determined in a radiometric assay using purified, recombinant enzyme and ATP at a concentration of 1 uM. All compounds were serially diluted in 100% DMSO. The kinase reaction was incubated for 1 hour at room temperature, and the reaction was terminated by the addition of PBS. IC₅₀ values were subsequently determined using sigmoidal dose-response curve fit (variable slope). All of the compounds tested had an IC₅₀ against PI3K of 50 μM or less. Typically the IC₅₀ against PI3K was 5-500 nM.

### (ii) Cellular Proliferation Inhibition

Cells were seeded at optimal density in a 96 well plate and incubated for 4 days in the presence of test compound. Alamar Blue™ was subsequently added to the assay medium, and cells were incubated for 6 hours before reading at 544 nm excitation, 590 nm emission. EC₅₀ values were calculated using a sigmoidal dose response curve fit. All the compounds tested had an EC₅₀s of 50 uM or less in the range of cell lines utilized.

### Example 10

### Tablet Composition

Tablets, each weighing 0.15 g and containing 25 mg of a compound of the invention were manufactured as follows:

Composition for 10,000 tablets
Compound of the invention (250 g)

### Lactose (800 g)

Corn starch (415 g)
Talc powder (30 g)
Magnesium stearate (5 g)

The compound of the invention, lactose and half of the corn starch were mixed. The mixture was then forced through a sieve 0.5 mm mesh size. Corn starch (10 g) is suspended in warm water (90 ml). The resulting paste was used to granulate the powder. The granulate was dried and broken up into small fragments on a sieve of 1.4 mm mesh size. The remaining quantity of starch, talc and magnesium was added, carefully mixed and processed into tablets.

### Example 11

### Injectable Formulation

| Compound of the invention | 200 | mg |
|---|---|---|
| Hydrochloric Acid Solution 0.1M or | 4.0 to 7.0 | |
| Sodium Hydroxide Solution 0.1M q.s. to pH | | |
| Sterile water q.s. to | 10 | ml |

The compound of the invention was dissolved in most of the water (35°-40° C.) and the pH adjusted to between 4.0 and 7.0 with the hydrochloric acid or the sodium hydroxide as appropriate. The batch was then made up to volume with water and filtered through a sterile micropore filter into a sterile 10 ml amber glass vial (type 1) and sealed with sterile closures and overseals.

### Example 12

### Intramuscular Injection

| | | |
|---|---|---|
| Compound of the invention | 200 | mg |
| Benzyl Alcohol | 0.10 | g |
| Glycofurol 75 | 1.45 | g |
| Water for injection q.s to | 3.00 | ml |

The compound of the invention was dissolved in the glycofurol. The benzyl alcohol was then added and dissolved, and water added to 3 ml. The mixture was then filtered through a sterile micropore filter and sealed in sterile 3 ml glass vials (type 1).

### Example 13

### Syrup Formulation

| | | |
|---|---|---|
| Compound of invention | 250 | mg |
| Sorbitol Solution | 1.50 | g |
| Glycerol | 2.00 | g |
| Sodium benzoate | 0.005 | g |
| Flavour | 0.0125 | ml |
| Purified Water q.s. to | 5.00 | ml |

The compound of the invention was dissolved in a mixture of the glycerol and most of the purified water. An aqueous solution of the sodium benzoate was then added to the solution, followed by addition of the sorbital solution and finally the flavour. The volume was made up with purified water and mixed well.

1. A compound which is a pyrimidine of formula (I): 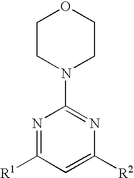
wherein
R¹ is a group —NR—(CHR)ₘ—X;
R² is a substituted indolyl group;
R is H or C₁-C₆ alkyl;
m is 1, 2, 3 or 4; and
X is a pyridyl ring;
or a pharmaceutically acceptable salt thereof.

2. A compound according to claim 1 wherein the pyrimidine is of formula (Ia): 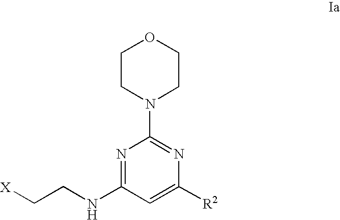
wherein R² and X are as defined in claim 1.

3. A compound according to claim 1 wherein R² is an indol-4-yl group which is substituted at the 5-position by halo or at the 6-position by halo, CN, CF₃, —CONH₂, —SO₂NMe₂ or —SO₂Me.

4. A compound which is selected from:
[6-(6-Fluoro-1H-indol-4-yl)-2-morpholin-4-yl-pyrimidin-4-yl]-(2-pyridin-3-yl-ethyl)-amine;
4-[2-morpholin-4-yl-6-(2-pyridin-3-yl-ethylamino)-pyrimidin-4-yl]-1H-indole-6-sulfonic acid dimethylamide;
[6-(5-Fluoro-1H-indol-4-yl)-2-morpholin-4-yl-pyrimidin-4-yl]-(2-pyridin-3-yl-ethyl)-amine;
4-[2-Morpholin-4-yl-6-(2-pyridin-3-yl-ethylamino)-pyrimidin-4-yl]-1H-indole-6-carbonitrile;
[6-(6-Methanesulfonyl-1H-indol-4-yl)-2-morpholin-4-yl-pyrimidin-4-yl]-(2-pyridin-3-yl-ethyl)-amine; and
4-[2-Morpholin-4-yl-6-(2-pyridin-3-yl-ethylamino)-pyrimidin-4-yl]-1H-indole-6-carboxylic acid amide;
[6-(2-Trifluoromethyl-1H-indol-4-yl)-2-morpholin-4-yl-pyrimidin-4-yl]-(2-pyridin-3-yl-ethyl)-amine;
[6-(2-Cyano-1H-indol-4-yl)-2-morpholin-4-yl-pyrimidin-4-yl]-(2-pyridin-3-yl-ethyl)-amine;
and the pharmaceutically acceptable salts thereof.

5. A pharmaceutical composition which comprises a pharmaceutically acceptable carrier or diluent and, as an active ingredient, a compound as defined in claim 1.

6-9. (canceled)

10. A method of treating a disease or disorder arising from abnormal cell growth, function or behaviour associated with PI3 kinase, which method comprises administering to a patient in need thereof a compound as defined in claim 1.

11. A method according to claim 10 herein the disease or disorder is selected from cancer, immune disorders, cardiovascular disease, viral infection, inflammation, metabolism/endocrine function disorders and neurological disorders.
